(19) United States
(12) Patent Application Publication
(10) Pub. No.: US 2016/0287723 A1
(43) Pub. Date: Oct. 6, 2016

(54) Method of Fabricating Anticancer Drug Having Doxorubicin Bonded with Gold Nanoparticles

(71) Applicant: Institute of Nuclear Energy Research, Atomic Energy Council, Executive Yuan, R.O.C., Taoyuan City (TW)

(72) Inventors: Chun-Chia Cheng, Taoyuan City (TW); Tsai-Yueh Luo, Taoyuan City (TW)

(21) Appl. No.: 14/672,711

(22) Filed: Mar. 30, 2015

(51) Int. Cl. A61K 47/48; A61K 31/704

(52) U.S. Cl. CPC A61K 47/48869; A61K 31/704

(57) ABSTRACT

Albumin and doxorubicin are bonded through reaction. The bonded albumin and doxorubicin are added with a gold-nanoparticles solution for reaction. After being purified with water in a column, the liquid phase of gold-nanoparticles is changed into a phosphate buffer for obtaining an anticancer drug having doxorubicin bonded with gold nanoparticles. Thus, with the high biocompatibility and the big surface areas, gold nanoparticles are used in biological applications and used as carriers for increasing drug accumulation on tumor. The bonding between gold nanoparticles and doxorubicin increases stability of doxorubicin. Besides, doxorubicin has tumor-killing effect for cancer therapy.

### FIELD OF THE INVENTION

The present invention relates to a method for bonding gold nanoparticles to doxorubicin (DOX); more particularly, relates to using gold nanoparticles with high biocompatibility and big surface areas in biological applications and using gold nanoparticles as carriers to increase drug accumulation ON tumor, where gold nanoparticles are bonded to DOX for increasing stability of DOX, improving cytotoxic effect to tumor and enhancing effective therapy of cancer.

### BACKGROUND OF THE INVENTION

Nanotechnologies can be used in many industries, including biomedical pharmaceutical industries, which often have unexpected effects on changing physical properties of drug.

Generally, nanotechnologies refer to particles having diameters around 1˜100 nanometers (nm), where the particles can be modified into a variety of shapes, such as round, long cylindrical or square shapes, to meet different fields of applications. Currently, diagnosis or treatment of cancer can use nanomaterials, such as liposomes, micelles and gold nanoparticles, as drug carriers (Liu, G., Swierczewska, M., Lee, S. and Chen, X., Functional Nanoparticles for Molecular Imaging Guided Gene Delivery. Nano Today. 2012, 5, 524-539) for achieving great drug efficacy. Based on recent studies, nanotechnologies can improve enhanced permeability and retention (EPR) effect in organism. That is, nanomaterials (<200 nm) can traverse incomplete vascular endothelial cells to be gathered in cancer tissues. Vice versa, although small-molecule drugs may also enter cancer tissues, but may fade easily. Hence, nanomaterials can be used as drug carriers for diagnosis or treatment of cancer.

At present, for synthesizing gold nanoparticles, tetrachloroauric acid (HAuCl₄) is usually used to be added into a reducing agent (e.g. sodium boron hydride (NaBH₄)) for dissociate gold ions (Au³⁺→Au). Then, a stabilizing agent is added (e.g. albumin) to be adhered on surface of the gold ions to prevent accumulation of the gold ions. Or, sodium citrate (C₆H₅Na₃O₇) can be used as a reducing agent as well as a stabilizing agent (Baptista, P., Pereira, E., Eaton, P., Doria, G., et al., Gold nanoparticles for the development of clinical diagnosis methods. Anal Bioanal Chem. 2008, 391, 943-950.)

Gold nanoparticles can be bonded with different styles of compounds, such as DNAs, proteins or drugs, on their surfaces. Besides, due to their good biocompatibilities and big surface areas, gold nanoparticles are suitable for biological applications and for increasing drug efficacy. Previous studies have confirmed that gold nanoparticles can indeed enhance effects of anticancer drugs (Zhang, Z., Jia, J., Lai, Y., Ma, Y., et al., Conjugating folic acid to gold nanoparticles through glutathione for targeting and detecting cancer cells. Bioorg Med Chem. 2010, 18, 5528-5534; Chithrani, D. B., Jelveh, S., Jalali, F., van Prooijen, M., et al., Gold nanoparticles as radiation sensitizers in cancer therapy. Radiat Res. 2010, 173, 719-728; Chanda, N., Kan, P., Watkinson, L. D., Shukla, R., et al., Radioactive gold nanoparticles in cancer therapy: therapeutic efficacy studies of GA-198AuNP nanoconstruct in prostate tumor-bearing mice. Nanomedicine. 2010, 6, 201-209; Lim, Z. Z., Li, J. E., Ng, C. T., Yung, L. Y. and Bay, B. H., Gold nanoparticles in cancer therapy. Acta Pharmacol Sin. 2011, 32, 983-990; Jain, S., Hirst, D. G. and O'Sullivan, J. M., Gold nanoparticles as novel agents for cancer therapy. Br J Radiol. 2012, 85, 101-113.).

Synthesis and purification of gold nanoparticles are well-known technologies; however, the use of gold nanoparticles in cancer treatment is one of the on focus developments in recent years. Because gold nanoparticles may be easily reacted with sulfur groups to form stable ionic bonds on their surfaces, albumin had mentioned to be used to stabilize gold nanoparticles for cancer treatment. But, using albumin as a link for bonding gold nanoparticles to DOX is not found.

Hence, the prior arts do not fulfill all users' requests on actual use.

### SUMMARY OF THE INVENTION

The main purpose of the present invention is to bond gold nanoparticles to DOX, where gold nanoparticles with high biocompatibility and big surface areas are used in biological applications and used as carriers for increasing drug accumulation on tumor; and the present invention increases stability of DOX, improves cytotoxic effect to tumor and enhances effective therapy of cancer.

To achieve the above purpose, the present invention is a method of fabricating an anticancer drug having DOX bonded with gold nanoparticles, comprising steps of: (a) obtaining DOX to be added with five times of bone morphogenetic proteins (BMPs) to process reaction for 22-26 hours (hr) in 100% dimethylformamide (DMF) to bond amine groups of DOX with maleimide; processing purification by using a RP18 column to obtain DOX-maleimide; dissolving an albumin in a carbonate-bicarbonate buffer with 10 times of 2-iminothiolane hydrochloride added to process reaction for 0.5˜1.5 hrs; after removing excess 2-iminothiolane hydrochloride through a G25 column, adding 5 times of DOX-maleimide with phosphate buffered saline (PBS) to process reaction for 1.5˜2.5 hrs for obtaining a solution of albumin-DOX having albumin bonded to DOX; and processing purification with water through a G25 column; and (b) obtaining the solution of albumin-DOX to process reaction with dithiothreitol (DTT) in pure water for 0.5˜1.5 hrs to break disulfide bond of albumin; after removing DTT through a G25 column, processing reaction by adding a gold-nanoparticles solution; after processing purification by using a membrane, turning a liquid phase of the reacted gold-nanoparticles solution into a phosphate buffer to obtain an anticancer drug of gold-nanoparticles-DOX (GNPs-DOX). Accordingly, a novel method of fabricating an anticancer drug having DOX bonded with gold nanoparticles is obtained.

### BRIEF DESCRIPTION OF THE DRAWINGS

The present invention will be better understood from the following detailed description of the preferred embodiment according to the present invention, taken in conjunction with the accompanying drawings, in which

FIG. 1 is the flow view showing the preferred embodiment according to the present invention;

FIG. 2 is the view showing the mass spectrometry of albumin-doxorubicin(DOX);

FIG. 3 is the view showing the intensities obtained after being purified in the G25 columns; and

FIG. 4 is the flow view showing the bonding of gold nanoparticle to albumin-doxorubicin.

### DESCRIPTION OF THE PREFERRED EMBODIMENT

The following description of the preferred embodiment is provided to understand the features and the structures of the present invention.

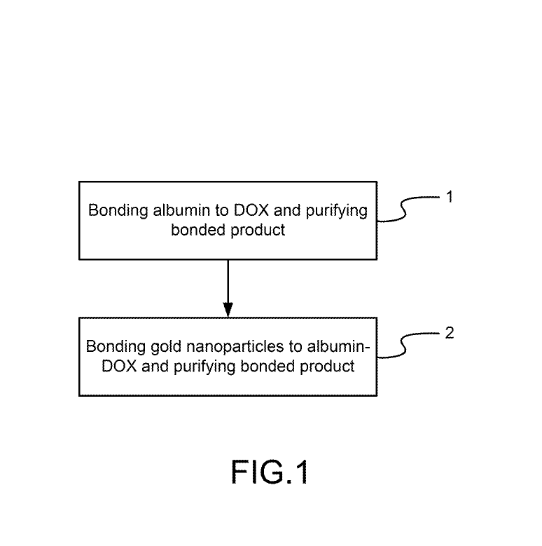

Please refer to FIG. 1-FIG. 4, which are a flow view showing a preferred embodiment according to the present invention; a view showing mass spectrometry of albumin-DOX; a view showing intensities obtained after being purified in G25 columns; and a flow view showing bonding of gold nanoparticle to albumin-DOX. As shown in the figures, the present invention is a method of fabricating an anticancer drug having DOX bonded with gold nanoparticles, comprising the following steps:

(a) Bonding albumin to DOX and purifying bonded product 1: DOX is obtained to be added with five times of bone morphogenetic proteins (BMPs) to process reaction for 24 hours (hr) in 100% dimethylformamide (DMF) to bond amine groups of DOX with maleimide. Then, an RP18 column is used for purification to obtain DOX-maleimide having a molecular weight of 693. Meanwhile, albumin is obtained to be dissolved in a 0.05M buffer of carbonate-bicarbonate (pH 8.0) with 10 times of 2-iminothiolane hydrochloride added to process reaction for 1 hr. After removing excess 2-iminothiolane hydrochloride through a G25 column, five times of DOX-maleimide is added with phosphate buffered saline (PBS) to process reaction for 2 hrs for obtaining albumin-DOX with completion of bonding albumin to DOX. Then, the completed product is purified with water through a G25 column.

(b) Bonding gold nanoparticles to albumin-DOX and purifying bonded product 2: Twenty milligrams of albumin-DOX is obtained to process reaction with 100 millimole (mM) of dithiothreitol (DTT) for 1 hr in pure water to break disulfide bond of albumin. After removing DTT through a G25 column, 2 milliliters of a 0.4 mM solution of gold nanoparticles is added to process reaction. After processing purification through a 100 kDa Amicon membrane, a liquid phase of the reacted solution is transformed into a phosphate buffer for obtaining an anticancer drug of gold-nanoparticles-DOX (GNPs-DOX).

Thus, a novel method of fabricating an anticancer drug having DOX bonded with gold nanoparticles is obtained.

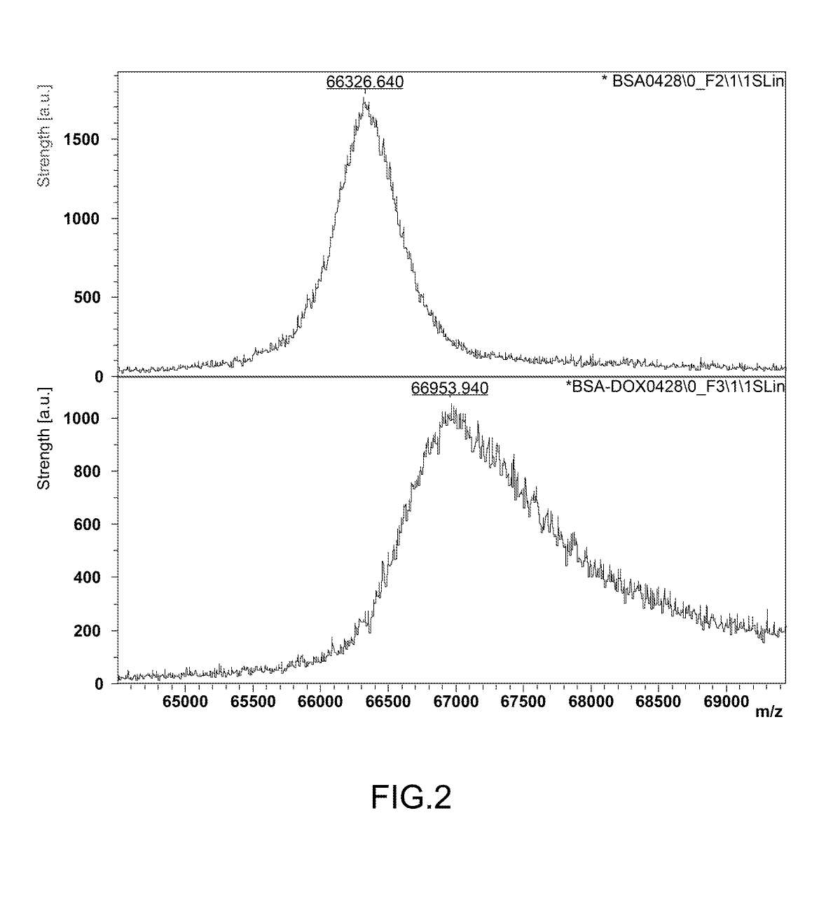

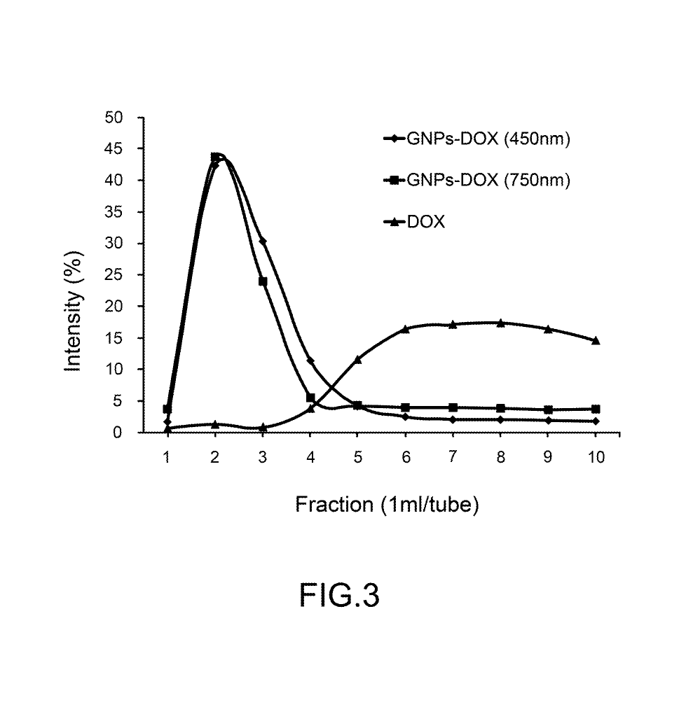

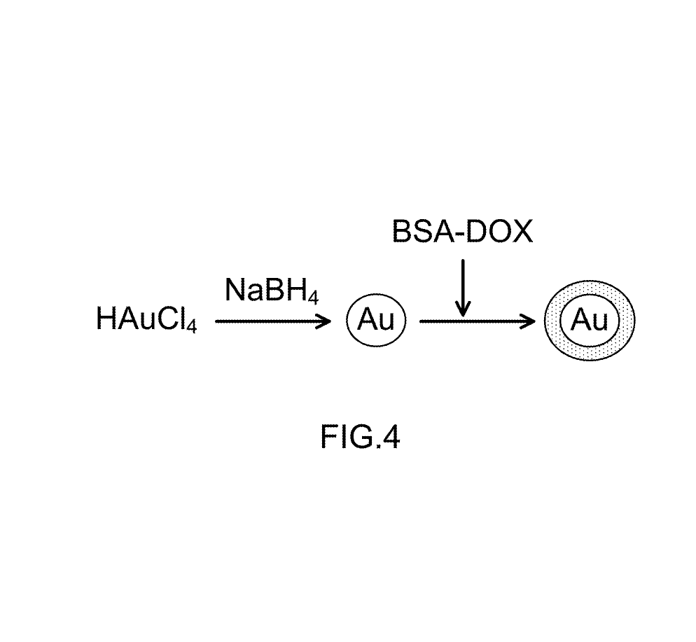

In a state-of-use, the present invention uses bovine serum albumin (BSA) as a stabilizer to be bonded to DOX. After completing the bonding of BSA to DOX in step (a), mass spectrometry is used for analyzing bonding efficiency. As shown in FIG. 2, BSA has a molecular weight of 66.3 kDa and, after BSA is bonded to DOX, BSA-DOX has a molecular weight of 66.9 kDa, which shows that DOX is successfully bonded with BSA in a 1:1 ratio. Then, as shown in FIG. 3, a solution of gold nanoparticles is added to process reaction for 60 minutes. As shown in FIG. 4, after processing purification with a G25 column, the solution is transformed into a phosphate buffer to obtain an anticancer drug of GNPs-DOX.

To sum up, the present invention is a method of fabricating an anticancer drug having DOX bonded with gold nanoparticles, where gold nanoparticles with high biocompatibility and big surface areas are used in biological applications and used as carriers for accumulating drug on tumor; and DOX is effectively used in cancer therapies by inhibiting tumor.

The preferred embodiment herein disclosed is not intended to unnecessarily limit the scope of the invention. Therefore, simple modifications or variations belonging to the equivalent of the scope of the claims and the instructions disclosed herein for a patent are all within the scope of the present invention.

1. A method of fabricating an anticancer drug having doxorubicin (DOX) bonded with gold nanoparticles, comprising steps of:
(a) obtaining an amine-containing drug to be added with five times molar equivalent of N-β-maleimidophenylacetic acid chloride (BMPS) to process reaction in 100% dimethylformamide (DMF) to bond maleimide to said amine-containing drug at amine groups; processing purification through a RP18 column; dissolving an albumin in a carbonate-bicarbonate buffer with 10 times molar equivalent of 2-iminothiolane hydrochloride added to process reaction; after removing excess 2-iminothiolane hydrochloride through a G25 column, adding 5 times molar equivalent of said maleimide-bonded amine-containing drug with phosphate buffered saline (PBS) to process reaction to bond said albumin to said amine-containing drug; and processing purification with water through a G25 column; and
(b) obtaining said albumin-bonded amine-containing drug to process reaction with dithiothreitol (DTT) in pure water to break disulfide bond of albumin; after removing DTT through a G25 column, processing reaction by adding a gold-nanoparticles solution; after processing purification by using a membrane, changing a liquid phase of said reacted gold-nanoparticles into a phosphate buffer to obtain an anticancer drug bonded with gold nanoparticles, wherein said amine-containing drug is DOX and said anticancer drug is gold-nanoparticles-DOX (GNPs-DOX).

2. The method according to claim 1, wherein, in step (a), said amine-containing drug, DOX, is added with said N-β-maleimidophenylacetic acid chloride (BMPS) to process reaction for 22˜26 hours (hr).

3. The method according to claim 1, wherein, in step (a), said albumin is selected from a group consisting of bovine serum albumin (BSA) and human serum albumin (HBS).

4. The method according to claim 1, wherein, in step (a), said albumin is dissolved in said carbonate-bicarbonate buffer with 10 times molar equivalent of 2-iminothiolane hydrochloride added to process reaction for 0.5˜1.5 hrs.

5. The method according to claim 1, wherein, in step (a), said amine-containing drug bonded with maleimide, DOX-maleimide, is added with PBS to process reaction for 1.5˜2.5 hrs.

6. The method according to claim 5, wherein said DOX-maleimide has a molecular weight of 693.

7. The method according to claim 1, wherein, in step (b), said amine-containing drug bonded with said albumin, albumin-DOX, is obtained to process reaction with DTT for 0.5˜1.5 hrs.

8. The method according to claim 1, wherein said carbonate-bicarbonate buffer has a pH value of 6˜10.

9. (canceled)
